(19) United States
(12) Patent Application Publication
(10) Pub. No.: US 2023/0390182 A1
(43) Pub. Date: Dec. 7, 2023

(54) PROCESS FOR OBTAINING AN AQUEOUS EXTRACT OF LAVENDER, COMPOSITIONS COMPRISING SUCH AN EXTRACT, AND THEIR COSMETIC USES

(71) Applicant: ISP INVESTMENTS LLC., Wilmington, DE (US)

(72) Inventors: Elodie OGER, ANTIBES (FR); Catherine GONDRAN, BELLENAVES (FR); Florian LABARRADE, ANTIBES (FR)

(73) Assignee: ISP INVESTMENTS LLC, Wilmington, DE (US)

(21) Appl. No.: 17/795,389

(22) PCT Filed: Jan. 26, 2021
(86) PCT No.: PCT/EP2021/051734

§ 371 Date: Aug. 14, 2023

(30) Foreign Application Priority Data
Feb. 4, 2020 (FR) FR2001107

(51) Int. Cl. A61K 8/9789; A61Q 19/00

(52) U.S. Cl. CPC A61K 8/9789; A61Q 19/00; A61K 2800/10; A61K 2800/80

(57) ABSTRACT

The invention relates to a process for obtaining an aqueous extract of lavender enriched with small RNAs of a maximum length of 150 nucleotides, with sugars, with phenolic compounds, with organic acids and devoid of DNA, obtained by the following process: the lavender aerial parts are brought into contact with water; phytic acid is added at a pH of between 10 and 11; the pH of the mixture obtained is then adjusted to a value of between 6 and 8, the mixture obtained is purified. The invention also relates to cosmetic compositions comprising such an extract and their cosmetic uses for protecting the skin from external aggression and oxidation, combating the signs of skin ageing, increasing photoprotection, lightening the skin, improving hydration, reinforcing the barrier function, soothing the skin or improving the biological mechanisms associated with skin repair during the night.

### TECHNICAL FIELD

The invention relates to the field of cosmetics and more particularly to active ingredients of natural origin used in the preparation of cosmetic formulations to improve the appearance of the skin or protect the skin.

The invention relates to a process for obtaining an aqueous extract of lavender as well as to the extract enriched with small RNAs, with sugars, with phenolic compounds, with organic acids obtained by the process, to the cosmetic compositions comprising such extracts, and to their cosmetic uses for skincare, for care of the scalp and the appendages, and more particularly for protecting the skin from external aggressions and oxidation, for combating the signs of skin ageing, for increasing photoprotection, for lightening the skin, for improving skin hydration, for reinforcing the barrier function, for soothing the skin, or for improving the biological mechanisms associated with skin repair during the night.

### TECHNICAL BACKGROUND OF THE INVENTION

The plants of the genus Lavandula, commonly known as lavender, form a group of 47 species. Among the best-known and most widely used species are Lavandula angustifolia, also known as true lavender, a wild species native to Provence (south-eastern France), and lavandin, a hybrid resulting from a cross between true lavender and spike lavender.

True lavender is widely used for the production of essential oils in Europe, North Africa, the Middle East and Asia, particularly for the perfume industry.

The essential oil (EO) is generally obtained by hydrodistillation. The flowers, dried beforehand, are subjected to a stream of steam, which carries away the volatile or soluble constituents and is then recondensed to obtain a hydrolate (or floral water) and a supernatant comprising the lipidic parts of the plant and constituting the essential oil.

Lavender EOs are very fragrant and contain mostly volatile monoterpenes, such as linalool and linalyl acetate. In true lavender EO, both are present at about 30%. Lavender EO is particularly known for its sedative, pain-relieving, analgesic, anti-inflammatory, antiseptic and antibacterial properties. It also has an antispasmodic and decongestant effect and is indicated for soothing skin conditions and promoting healing. Lavender EO is also indicated to improve sleep disorders and various tensions (anxiety, stress, etc.).

Lavender floral waters contain less than a few percent of volatile organic compounds similar to those in the essential oil, as well as the plant's water-soluble compounds. Floral water has the same properties as lavender essential oil, but more attenuated due to the lower concentrations of terpene compounds.

The very limited use of lavender essential oils and floral waters in cosmetics is partly due to the majority presence of terpene molecules, known for their irritating effects on the skin. Linalool, for example, has been recognised as potentially allergenic and the use of lavender essential oil is not recommended for pregnant women and children under 12 years old.

Most of the other lavender extraction methods described in the prior art use organic solvents of the apolar type, which make it possible to extract mainly volatile odorant compounds (CN10338583, JP11199469) or a supercritical fluid (KR2015042999). In the latter case, the extract obtained is rich in polyphenols and flavonoids, but does not contain other compounds of interest, such as amino acids, organic acids, or proteins. Moreover, phenolic acids are not extracted by this type of technique, as these molecules are extracted mainly in a polar solvent and ideally with water.

Consumers of cosmetic products want to use formulas that are as natural as possible, yet still as effective as or more effective than synthetic products.

Despite the various anti-ageing cosmetic products already on the market, there is an ever-increasing need for new, effective cosmetic ingredients of natural origin.

One problem that the invention proposes to solve is to offer a new aqueous extract of lavender that meets the requirements of the current cosmetic market with regard to naturalness criteria and yet has an exceptional biological efficacy.

Another problem that the invention proposes to solve is to propose a new aqueous extract of lavender that does not have the disadvantages of currently known extracts, namely a strong odour, an instability of EO in formulas for topical application, or an irritant or allergenic character due to the presence of terpene molecules such as linalool.

Another problem that the invention proposes to solve is to offer a new aqueous extract of lavender enriched with compounds known to be effective on the skin, such as small RNAs, sugars, phenolic compounds and organic acids.

The inventors have developed a new lavender flower extract specifically enriched with small RNAs, sugars, phenolic compounds and organic acids, and without the disadvantages of the cited prior art processes, such as the use of potentially toxic detergents and solvents in cosmetics.

The extract thus obtained can be used in cosmetics for skincare, care of the scalp and skin appendages, and more particularly to protect the skin from external aggressions and oxidation, to fight against the signs of skin ageing, to increase photoprotection, to lighten the skin, to improve skin hydration, to reinforce the barrier function or to soothe the skin.

### SUMMARY OF THE INVENTION

The invention relates firstly to a process for obtaining an aqueous extract of lavender aerial parts, comprising the following steps
a) bringing the lavender aerial parts into contact with water;
b) adding phytic acid at a concentration of between 1 and 10 mM to the mixture obtained in a) at a pH of between 10 and 11;
c) then adjusting the pH of the mixture obtained in b) to a value of between 6 and 8;
d) purifying the mixture obtained in c) so as to eliminate the residual solid plant matter and obtain a purified aqueous crude extract; and
e) checking the pH and readjusting it if necessary to a value between 6 and 8, preferably between 6 and 6.5.

Furthermore, the invention relates secondly to an aqueous extract of lavender aerial parts enriched with small RNAs with a length of at most 150 nucleotides, with sugars, with phenolic compounds and with organic acids, free of DNA, obtainable by the process of one of claims 1 to 5, wherein the extract comprises, by weight of the total weight of the extract, 10 to 30 g/kg of dry weight, containing 2 to 10 g/kg of sugars, 100 to 1500 mg/kg of organic acids, 500 to 2000 mg/kg of phenolic compounds and 40 to 200 mg/kg of low molecular weight RNA with a length of at most 150 nucleotides.

The invention relates thirdly to a cosmetic composition comprising, as an active ingredient, an effective amount of the extract of either claim 6 or 7, and a physiologically acceptable medium.

The invention relates fourthly to the cosmetic use of the composition of the invention for skincare, for care of the scalp and the skin appendages, more particularly for protecting the skin from external aggressions and oxidation, combating the signs of skin ageing, increasing photoprotection, lightening the skin, improving skin hydration, strengthening the barrier function, soothing the skin, or for improving the biological mechanisms associated with skin repair during the night.

### BRIEF DESCRIPTION OF THE DRAWINGS

The invention and its advantages will be better understood from the following description and non-limiting embodiments, illustrated with reference to the attached figures, in which:

FIG. 1 shows the characterisation of organic acids by HPLC-MS analysis

FIG. 2 shows an analysis of low molecular weight RNA by the Bioanalyser 2100. A: lavender extract obtained according to the process of the invention—B: conventional lavender extract.

### DETAILED DESCRIPTION OF THE INVENTION

### Definitions

All terms used in this description have the most widely known meaning unless otherwise stated. For the purposes of the invention the following terms are defined as follows:

“Lavender” refers to all species of the genus Lavandula, as well as their hybrids (such as lavandin).

“Aerial parts” means the stems and flowers of lavender. The seeds are included in the “aerial parts” within the meaning of the invention.

In the course of this description, the terms “aerial parts” and “flowers” will be used interchangeably to refer to the flowers and the thinner stems bearing the flowers.

The term “small RNAs” or “low molecular weight RNAs”, or “small RNAs of a length of at most 150 nucleotides” means non-coding RNAs (ribonucleic acids) of small molecular weight, of a length of at most 150 nucleotides, such as all types of small non-messenger RNAs, single and/or double stranded, for example micro-RNAs, interfering RNAs, introns, small nuclear RNAs or any RNA fragment. Electrophoretic analysis shows that the small RNAs present in the lavender extract of the invention have various molecular weights of approximately 30 to 150 nucleotides.

“Organic acids” means α-hydroxy acids (or AHAs), i.e. carboxylic acids derived from fruit or plant sugars, such as glycolic, malic, citric, tartaric, succinic and uronic acids.

“Phenolic compounds” (or polyphenols) means molecules of plant origin that have an aromatic ring bearing one or more hydroxyl groups, such as phenolic acids, flavonoids or their derivatives. Polyphenolic compounds are known to be powerful antioxidant molecules.

“Sugars” mean monosaccharides, especially glucose and fructose, as well as oligo- and polysaccharides.

“Phytomolecules of interest” means all the molecules present in the lavender extract of the invention and, in particular, small RNAs of a maximum length of 150 nucleotides, sugars, phenolic compounds and organic acids.

When a range of values is described, the limits of that range should be understood as explicitly including all intermediate values in the range. For example, a range of values between 1% and 10% should be understood to include 1%, 2%, 3%, 4%, 5%, 6%, 7%, 8%, 9%, and 10%, and also all decimal values between 1% and 10%.

Numerical percentage values are percentages by weight, i.e. the weight of a compound in relation to the total weight of the intended mixture, unless otherwise specified.

The compositions described in the present application may “comprise”, “consist of” or “consist substantially of” the essential compounds or optional ingredients.

“Consist substantially of” means that the composition or component may include additional ingredients, but only if the additional ingredients do not alter the basic or novel characteristics of the composition or use described in the present application.

A “physiologically acceptable medium” means a vehicle that is suitable for coming into contact with the outer layers of the skin or mucous membranes, without toxicity, irritation, undue allergic response and the like or intolerance reaction, and proportionate to a reasonable benefit/risk ratio.

“Topical application” means applying or spreading the aqueous extract enriched with small RNAs of a maximum length of 150 nucleotides, sugars, phenolic compounds and organic acids according to the invention, or a composition containing it, on the surface of the skin or a mucous membrane.

“Skin” refers to the skin of the face, in particular the eye area and mouth, the nose, the forehead, the neck, the hands, but also the skin of the whole body.

“Scalp” means the skin covering the skull, including the hair follicles and the inter-follicular skin spaces.

“Skin appendages” refers to the products of hair follicles (hair and body hair) and also nails, which are rich in keratin.

The expression “strengthening the barrier function” means improving the skin's protective properties against external aggressions (UV radiation, visible or infrared light, pollution, microorganisms, etc.).

The expression “soothing the skin” means reducing irritation reactions that manifest themselves as a feeling of discomfort, which may be accompanied by redness.

The expression “lightening the skin” means reducing the intensity of skin colour linked to the melanin content of the epidermis, either homogeneously or in a localised manner by acting on pigmentary disorders, such as senescence spots or senile lentigo.

“Effective quantity” means the minimum quantity of extract according to the invention which is necessary to obtain at least one of the biological activities sought, in particular to demonstrate antioxidant activity against reactive oxygen species, to increase melatonin or decrease melanin, or any other biological marker studied, without this quantity being toxic.

“Skin hydration” refers to the water content and distribution in the upper layers of the epidermis.

“Improved skin hydration” refers to any improvements of changes in the external appearance of the skin due to dehydration, such as dryness, tightness and discomfort, whether this condition is related to internal or external factors, such as adverse environmental conditions.

“Signs of skin ageing” means all changes in the external appearance of the skin due to ageing such as, for example, wrinkles and fine lines, cracks, bags under the eyes, dark circles, withering, loss of elasticity, firmness and/or tone of the skin, but also all internal modifications of the skin which do not systematically result in a modified external appearance such as, for example, thinning of the skin, or all internal degradations of the skin resulting from environmental stresses such as pollution and solar radiation including UV radiation.

“Signs of skin ageing” also includes pigmentary disorders such as senile lentigo or solar lentigo.

“External aggressions” means solar radiation, including visible light, UV and infrared radiation, pollution, which may come from the ambient atmosphere outside or inside homes and includes particles of various sizes (10 μm for PM10, 2.5 μm for PM 2.5, or less than 100 nm for ultrafine particles) as well as several chemical elements (volatile organic compounds, polycyclic aromatic hydrocarbons, heavy metals, etc.).

“Improving the appearance of the skin” means that the skin texture appears finer, the luminosity more intense, and the skin tone more even.

It is understood that the invention relates to mammals and more particularly to human beings.

### Extraction Process

Classically described ribonucleic acid (RNA) extraction protocols use solvents unsuitable for cosmetic use (Zumbo, p. 2014 “Phenol-chloroform Extraction”, 2014). These methods aim to obtain completely purified nucleic acids (RNA or DNA or small RNAs), i.e. free of any other molecules of interest such as secondary metabolites, vitamins, sugars, peptides, etc., that may have beneficial effects on the skin and are therefore of cosmetic interest.

Also known is FR2831168, which describes a process for obtaining a plant extract rich in nucleic acids (DNA and/or RNA). The process uses cellulolytic enzymes.

Also known in the prior art are patent documents EP1723958 and WO03101376, which describe a composition for topical application comprising a synthetic double-stranded RNA oligonucleotide of known sequence and 12 to 40 nucleotides in length having an siRNA (short interfering) function.

Also known is document FR 1502361 (also published under number WO2017084958), which describes a process for obtaining an aqueous plant extract enriched with low molecular weight ribonucleic acids (RNA) for preparing cosmetic compositions. The process uses EDTA at a concentration of between 2 and 15 mM.

The use of phytic acid instead of EDTA has the following advantages: Unlike EDTA, phytic acid is a natural molecule found in the husk of seeds, such as cereals and legumes. Therefore, the use of phytic acid makes it possible to obtain a 100% natural lavender extract while maintaining a good extraction efficiency of the phytomolecules contained in the plant. The extraction efficiency of small RNAs also applies for other compounds present in lavender flowers, such as sugars, phenolic compounds, or organic acids, such as tartaric, malic or citric acid.

The invention thus relates firstly to an extraction process used to obtain an aqueous extract of the dried or fresh lavender aerial parts.

The extraction process of the invention makes it possible to obtain an extract rich in phytomolecules of cosmetic interest, such as small RNAs of a maximum length of 150 nucleotides, sugars, phenolic compounds and organic acids, avoiding the use of solvents which are not considered cosmetic solvents.

The process of the invention has a reduced impact on the environment.

In a first step a) of the process according to the invention, the lavender aerial parts are mixed with water. The water used is distilled water, demineralised water or water rich in mineral salts and/or trace elements. The water used is preferably distilled water.

Preferably the lavender aerial parts are the lavender flowers and the small stems carrying the flowers.

Preferably, the species of lavender used is Lavandula angustifolia or true lavender.

Preferably, the lavender aerial parts are in dried form.

Preferably, the lavender aerial parts are ground into a powder before being brought into the presence of water in step a). Grinding the lavender aerial parts is a mechanical action which allows for better extraction. Mechanical grinding to obtain the plant material in powder form, followed by alkaline lysis in the presence of phytic acid, promotes the complete destructuring of the cell membranes and in particular the nuclear membrane. Preferably, the previously ground lavender aerial parts in powder form are mixed with water in step a) in a plant material/water ratio of 3 to 20% w/w, more preferably in a ratio between 3 and 10%, for example in a ratio of 3%, 5% or 10% (w/w).

Phytic acid is then added in step b) to the mixture obtained in a). The pH in this step must be basic, between 10 and 11, and must therefore be adjusted, if necessary, by adding soda (NaOH). In step b) it is essential that the pH is basic, between 10 and 11. Preferably, the pH is adjusted to a value between 10.5 and 11. Indeed, this pH level, associated with the action of phytic acid, causes the destructuring of the cell membrane, including the nuclear membrane, the lysis of the cells and the denaturation of the DNA (the 2 strands of the double helix are separated). Phytic acid weakens and destructures the pecto-cellulosic membranes of plant cells by sequestering, by complexation, divalent ions such as calcium ions that form ionic bridges between the pectin molecules surrounding the cellulose microfibrils. This results in promoting the release of the cell content during the extraction process. The phytic acid treatment step is essential to enrich the extract in low molecular weight RNA and more generally to ensure a better extraction yield of other phytomolecules of interest; namely sugars, phenolic compounds and organic acids.

Monitoring of the pH shows that it remains basic and stabilises between 9 and 11 at the end of step b).

The extraction process makes it possible to enrich the final extract in low molecular weight RNAs through the use of an aqueous extraction solution containing a natural chelating agent such as phytic acid. Phytic acid is a molecule naturally present in the shells of seeds, such as cereals and legumes. Phytic acid is present as calcium salts or sometimes magnesium salts and plays an important role for the plant, for example, it is the main source of phosphorus.

Preferably, the phytic acid used is a phytic acid powder in the form of a sodium salt at a concentration preferably between 1 and 10 mM, preferably between 1 and 5 mM, and more preferably the concentration is 3 mM.

The invention works particularly well for a phytic acid concentration between 2 and 3 as described in Table 1 below. It can be seen that for only 2.25 mM phytic acid the same results are obtained as with 10 mM EDTA in terms of small RNA concentration, as well as a similar overall extraction yield. A concentration of 3 mM allows for optimum extraction efficiency of low molecular weight RNAs. The 3 mM concentration is also optimal for higher yields of other compounds of interest, such as sugars, phenolic compounds and organic acids. With a phytic acid concentration of 4.5 mM the extraction yield of low molecular weight RNAs is even higher, but the overall extraction yield drops.

TABLE 1
Low molecular weight RNA yield and overall extraction
yield as a function of phytic acid concentration

| Phytic Acid | Low molecular weight | Overall |
|---|---|---|
| Concentration | RNA concentration | extraction |
| (mM) | (mg/L) | yield (%) |
| Phytic acid 4.5 mM | 60 | 35 |
| Phytic acid 3 mM | 58 | 45 |
| Phytic acid 2.25 mM | 55 | 38 |
| Tetrasodium EDTA 10 mM | 56 | 40 |

Step b) of treatment with phytic acid preferably lasts at least 1 hour, at a temperature of between 20 and 80° C. During this step, the mixture obtained in a) is advantageously agitated.

Advantageously, diatomaceous earths can be added at the end of step b) to facilitate the subsequent separation of the solid residual plant material from the extract (soluble fraction) in the next step.

In a step c), the pH of the mixture obtained in b) is then adjusted to a value between 6 and 8.

The pH can be adjusted by adding a hydrochloric acid (HCl) solution or any other equivalent acid, compatible with cosmetic use. Acidification causes the DNA to undergo an abrupt renaturation (re-pairing of the strands of the double helix). However, chromosomal DNA, which is very long, does not manage to re-pair completely and forms insoluble entanglements. On the contrary, the small RNAs remain in solution. The DNA and small RNAs are thus separated into two distinct phases; a solid phase containing, among other things, the chromosomal DNA, and a liquid phase containing, among other things, the small RNAs. The pH adjustment step in step d) of the process according to the invention is an essential step for the optimal extraction of small RNAs, as well as other phytomolecules of interest; namely sugars, phenolic compounds and organic acids.

In a step d) the mixture obtained in c) is purified so as to eliminate the residual solid lavender aerial parts and recover the soluble part which constitutes the aqueous crude extract according to the invention. Any method known to the person skilled in the art may be used. For example, the mixture obtained in c) may be filtered on filters with a porosity greater than 30 μm so as to collect the filtrate. Preferably, the mixture obtained in c) is centrifuged at low speed, for example for at least 10 min at 4000 g, so as to sediment the residual plant material in the pellet and recover the aqueous crude extract in the supernatant.

In a step e) the pH is checked and readjusted to a value between 6 and 8. Preferably the pH is readjusted to a value between 6 and 6.5, even more preferably to a value of 6.5. The pH is readjusted by adding a hydrochloric acid (HCl) solution or any equivalent acid compatible with cosmetic use.

Indeed, a pH lower than 6 can lead to the precipitation of nucleic acids in general, and therefore of low molecular weight RNAs of a maximum length of 150 nucleotides. The step of adjusting the pH in step e) of the process according to the invention is an essential step in order to have optimal stability of the low molecular weight RNAs in the extract.

At the end of step e) a concentrated crude extract is obtained.

Advantageously, the readjustment of the pH in step e) is preceded by at least one filtration of the aqueous crude extract obtained in d). Preferably successive filtrations will be carried out by lowering the filtration threshold from 20 to 50 μm to a sterilising filtration of 0.1-0.3 μm.

The extract obtained in step e) can then be diluted in a physiologically acceptable solvent for cosmetic use, such that the dry weight is between 4 and 20 g/kg of dry extract in relation to the total weight of the diluted extract. This step improves the stability of the extract over time.

The invention relates secondly to an aqueous extract of lavender aerial parts enriched with small RNAs of a maximum length of 150 nucleotides, with sugars, with phenolic compounds, with organic acids and free of DNA, obtainable by the process described above. This extract does not contain DNA (deoxyribonucleic acid).

The invention also relates to an aqueous extract of lavender aerial parts enriched with small RNAs of a maximum length of 150 nucleotides, with sugars, with phenolic compounds and with organic acids, directly obtained by the process described above. This extract does not contain DNA (deoxyribonucleic acid).

Using the process of the invention according to steps a) to e), an aqueous concentrated crude extract of lavender of amber to dark amber colour with a dry weight of 10 to 30 g/kg, containing 2 to 10 g/kg of sugars, 100 to 1500 mg/kg of organic acids, 500 to 2000 mg/kg of phenolic compounds and 40 to 200 mg/kg of low molecular weight RNA with a length of maximum 150 nucleotides is obtained. Nevertheless, for aerial parts, in particular lavender flower of the species Lavandula angustifolia, the extracts obtained can present a significant variability depending on factors such as the location or year of harvesting, the season, the climatic conditions, the biotic stress, etc.

The extract thus obtained can then be diluted in a physiologically acceptable solvent for cosmetic use, so that the concentration of the extract is then adjusted to a dry weight of between 4 and 20 g/kg of dry extract based on the total weight of the diluted extract.

Illustrative and non-limiting examples of physiologically acceptable solvents are water, glycerol, ethanol, propanediol and its natural version called Zemea® produced from corn, butylene glycol, dipropylene glycol, ethoxylated or propoxylated diglycols, cyclic polyols or any mixture of these solvents.

Thus, the extract obtained can be diluted to obtain a final concentration of 50% plant-derived butylene glycol, or 50% plant-derived propanediol, or 30% plant-derived glycerine.

Preferably, the extract obtained by the process according to the invention is diluted in butylene glycol so that the diluted extract comprises a final butylene glycol concentration of 50%.

This so-called diluted extract comprises, by weight of the total weight of the extract, 4 to 20 g/kg dry extract, 0.5 to 10 g/kg sugars, 50 to 700 mg/kg organic acids, 50 to 1500 mg/kg phenolic compounds and 10 to 100 mg/kg low molecular weight RNA with a maximum length of 150 nucleotides.

A non-limiting example is a diluted extract of Lavandula angustifolia containing in particular sugars at a concentration of 1.7 g/kg, organic acids at a content of 570 mg/kg, 620 mg/kg of phenolic compounds, and 45 mg/kg of low molecular weight RNAs with a maximum length of 150 nucleotides.

On the contrary, lavender floral water and essential oil contain mainly terpene scent molecules and do not contain low molecular weight RNAs up to 150 nucleotides in length, nor sugars, nor phenolic compounds or organic acids.

The extract of the invention thus comprises a wide range of phytomolecules that may have beneficial effects on the skin, without presenting a risk of skin irritation or other health damage.

For example, the sugars actively participate in the hydration of the epidermal layers, and thus in the resistance to external aggressions, without showing any undesirable effect. The lavender extract of the invention contains more particularly mono- and polysaccharides, which are present neither in the floral water nor in the essential oil of lavender.

True lavender is a member of the Lamiaceae family, which has a particular metabolism known as CAM (Crassulacean Acid Metabolism). The plant stores organic acids and more particularly malic acid, citric acid and tartaric acid inside its cells. The process described in the invention makes it possible to extract these organic acids or AHAs. When applied to the skin, these AHAs reduce the cellular cohesion between the corneocytes, cause desquamation of the horny layers and thus stimulate cell renewal.

The lavender extract according to the invention is also enriched with phenolic compounds, such as phenolic acids. These water-soluble molecules known for their antioxidant activity contribute to the antioxidant and protective potential of the lavender extract of the invention.

A third aspect of the invention is a cosmetic composition comprising an effective amount of an aqueous extract enriched with small RNAs of a length of at most 150 nucleotides, with sugars, with phenolic compounds and with organic acids, obtained according to the invention, as an active ingredient, and a physiologically acceptable medium.

The aqueous extract enriched with small RNAs of a length of at most 150 nucleotides, with sugars, with phenolic compounds and with organic acids, obtained according to the invention, is advantageously used for the preparation of cosmetic compositions, as active ingredient.

Advantageously, the extract of lavender aerial parts according to the invention is added to the composition at a concentration of 0.05 to 5% by weight in relation to the total weight of the composition, preferably at a concentration of 0.1 to 2.5% by weight in relation to the total weight of the composition, and even more preferably at a concentration of 0.1 to 1.0% by weight in relation to the total weight of the composition. The composition usable according to the invention may be applied by any suitable route, in particular orally, or topically externally, and the formulation of the compositions will be adapted by the person skilled in the art.

Preferably, the compositions according to the invention are in a form suitable for topical application. These compositions must therefore contain a physiologically acceptable medium, i.e. compatible with the skin and the skin appendages, without any risk of discomfort during their application and must cover all suitable cosmetic forms.

The compositions for implementing the invention may in particular be in the form of an aqueous, hydroalcoholic or oily solution, an oil-in-water emulsion, water-in-oil emulsion or multiple emulsions; they may also be in the form of suspensions, or powders, suitable for application to the skin, mucous membranes, lips and/or hair.

These compositions may be more or less fluid and may also have the appearance of a cream, lotion, milk, serum, ointment, gel, paste or foam. They may also be in solid form, such as a stick, or may be applied to the skin in the form of an aerosol.

Examples of physiologically acceptable media commonly used in the envisaged field of application are adjuvants necessary for the formulation, such as solvents, thickeners, diluents, antioxidants, colouring agents, sun filters, self-tanning agents, pigments, fillers, preservatives, perfumes, odour absorbers, essential oils, vitamins, essential fatty acids, surfactants, film-forming polymers, etc.

In all cases, the person skilled in the art will ensure that these adjuvants and their proportions are chosen in such a way as not to impair the advantageous properties sought of the composition according to the invention. These adjuvants may, for example, correspond to 0.01 to 20% of the total weight of the composition. When the composition according to the invention is an emulsion, the fatty phase may represent from 5 to 80% by weight and preferably from 5 to 50% by weight in relation to the total weight of the composition. The emulsifiers and co-emulsifiers used in the composition are chosen from those conventionally used in the field under consideration. For example, they may be used in a proportion ranging from 0.3 to 30% by weight in relation to the total weight of the composition.

According to another advantageous embodiment of the invention, the aqueous lavender extract of the invention can be encapsulated or included in a cosmetic vector such as liposomes or any other nano capsule or microcapsule used in the field of cosmetics or adsorbed on powdery organic polymers, mineral supports such as talcs and bentonites.

Advantageously, the composition according to the invention may comprise, in addition to the active ingredient according to the invention, at least one other active agent having cosmetic effects similar and/or complementary to those of the invention.

For example, the additional active agent(s) may be selected from: anti-ageing, firming, lightening, moisturising, draining, microcirculation promoting, exfoliating, desquamating, extracellular matrix stimulating, energy metabolism activating, antibacterial, antifungal, soothing, anti-free radical, anti-UV, anti-acne, anti-inflammatory, anaesthetic, warm feeling inducing, cool feeling inducing, and slimming.

Such additional active agents may be selected from the groups comprising:
vitamin A and in particular retinoic acid, retinol, retinol propionate, retinol palmitate;
vitamin B3 and in particular niacinamide, tocopherol nicotinate;
vitamin B5, vitamin B6, vitamin B12, panthenol;
vitamin C, in particular ascorbic acid, ascorbyl glucoside, ascorbyl tetrapalmitate, magnesium and sodium ascorbyl phosphate;
vitamins E, F, H, K, PP, coenzyme Q10;
metalloproteinase inhibitors, or a TIMP activator;
DHEA, its precursors and derivatives;
amino acids such as arginine, ornithine, hydroxyproline, hydroxyproline dipalmitate, palmitoylglycine, hydroxylysine, methionine and its derivatives, N-acylated amino acid compounds;
natural or synthetic peptides, including di-, tri-, tetra-, penta- and hexapeptides and their lipophilic, isomeric and complexed derivatives with other species such as a metal ion (for example copper, zinc, manganese, magnesium, and others) As examples, mention may be made of the peptides commercially known as MATRIXYL®, ARGIRELINE®, CHRONOGEN™, LAMINIXYL IS™, PEPTIDE Q10™, COLLAXYL™ (patent FR2827170, ASHLAND®), PEPTIDE VINCI 01™ (patent FR2837098, ASHLAND®), PEPTIDE VINCI 02™ (patent FR2841781, ASHLAND®), ATPeptide™ (patent FR2846883, ASHLAND®) or the synthetic peptide of sequence Arg-Gly-Ser-NH2, marketed under the name ATPeptide™ by ASHLAND®;
Artemia salina extract, marketed under the name GP4G™ (FR2817748, ASHLAND®);
plant peptide extracts such as flax extracts (Lipigenin™, patent FR2956818, ASHLAND®), extracts of soya, spelt, vine, rapeseed, flax, rice, corn, peas;
yeast extracts, for example Dynagen™, (patent FR2951946, ASHLAND®) or Actopontine™ (patent FR2944526, ASHLAND®);
dehydroacetic acid (DHA);
phystosterols of synthetic or natural origin;
salicylic acid and its derivatives, alpha- and beta-hydroxy acids, silanols;
amino sugars, glucosamine, D-glucosamine, N-acetyl glucosamine, N-acetyl-D-glucosamine, mannosamine, N-acetyl mannosamine, galactosamine, N-acetyl galactosamine;
extracts of polyphenols, isoflavones, flavonoids, such as grape extracts, pine extracts, olive extracts;
lipids such as ceramides or phospholipids, oils of animal origin, such as squalene or squalane vegetable oils, such as sweet almond, copra, castor, jojoba, olive, rapeseed, peanut, sunflower, wheat germ, corn germ, soybean, cottonseed, alfalfa, alfalfa, cornflower, alfalfa oil, etc, cotton, alfalfa, poppy, pumpkin, evening primrose, millet, barley, rye, safflower, passionflower, hazelnut, palm, apricot kernel, avocado, calendula; ethoxylated vegetable oils, shea butter;
all UV screens and sun filters;
cyclic AMP and its derivatives, adenylate cyclase enzyme activators and phosphodiesterase enzyme inhibitors, Centella asiatica extract, asiaticoside and asiatic acid, methyl xanthines, theine, caffeine and its derivatives, theophylline, theobromine, forskolin, esculin and esculoside, ACE inhibitors, Val-Trp peptide, neuropeptide Y inhibitor, enkephalin, Ginkgo biloba extract, dioscorea extract, rutin, yerba mate extract, guarana extract, oligosaccharides, polysaccharides, carnitine, ivy extract, rockweed extract, hydrolysed Prunella vulgaris extract, hydrolysed extract of Celosia cristata, extract of Anogeissus leiocarpus, extract of Manihot utilissima leaves, palmitoylcarnitine, carnosine, taurine, elderberry extract, and seaweed extracts such as Palmaria Palmata extract.

The invention relates fourthly to the cosmetic use of a composition comprising the lavender extract of the invention for skincare, care of the scalp and the skin appendages, more particularly for protecting the skin from external aggressions and oxidation, for combating the signs of skin ageing, for increasing photoprotection, for lightening the skin, for improving skin hydration, for reinforcing the barrier function, for soothing the skin, or for improving the biological mechanisms associated with skin repair during the night.

The skin is an organ composed of several layers (dermis, epidermis and stratum corneum), which covers the entire surface of the body and ensures protective functions against external, sensory, immune, metabolic or thermoregulatory aggressions, or barrier functions, limiting dehydration.

In particular, the stratum corneum acts as a protective physical barrier, commonly referred to as the “skin's barrier function”. This function is of major importance in tissue homeostasis and protection from the external environment.

The appearance of the skin can be modified by internal alterations (intrinsic ageing, diseases and hormonal changes such as pregnancy) or external factors (environmental factors, such as pollution, sunlight, pathogens, temperature variations, etc.). All of these changes affect not only the skin, but also the keratinous appendages such as body hair, eyelashes, eyebrows, nails and hair on the head.

The extract of lavender aerial parts of the invention has been tested on key biological markers associated with night-time skin repair mechanisms. Mechanisms that occur in the skin at night include increased DNA repair, increased cell proliferation rate, increased skin temperature, increased skin blood flow, increased incidence of itching, and increased permeability of the skin barrier, leading to moisture loss (Matsui M. S. et al, Biological rhythms in the skin, Int. J. Mol. Sci. 2016, 17,801). In particular, the lavender aerial parts extract of the invention was evaluated on the rate of repair of pyrimidine dimers (CPD for Cyclobutane Pyrimidine Dimers). The day/night rhythm perceived at the level of the central nervous system includes the production of melatonin by the pineal gland (Slominski A. T. et al, Melatonin: A cutaneous perspective on its production, metabolism, and functions. J Invest Dermatol, 2018 March; 138(3):490-499). Melatonin is also produced locally in the skin from tryptophan. These functions help to decrease the level of reactive oxygen species (ROS) and reactive nitrogen species (RNS), respectively. Indeed, in addition to its direct role as a free radical scavenger, melatonin stimulates the production of antioxidant enzymes such as catalase and superoxide dismutase and is involved in DNA repair. By optimising the functioning of the mitochondria, melatonin also helps to increase the level of ATP produced by the cell. The effect of the extract of lavender aerial parts of the invention was evaluated on the melatonin level of the skin and on the expression of the enzyme AANAT (aralkylamine N-acetyltransferase or serotonin N-acetyltransferase or timezyme) which catalyses the N-acetylation of serotonin into N-acetylserotonin, the final step in the synthesis of melatonin. In addition, topical application of exogenous melatonin has been shown to have an effect on hair loss associated with androgenetic alopecia (Fisher TW, Topical melatonin for treatment of androgenetic alopecia, Int J Trichology, 2012 Oct.-Dec., 4(4):236-245).

The extract of lavender aerial parts of the invention has been shown to be effective in increasing melatonin production in cultured skin biopsies.

Changes to the barrier function result from external aggressions such as UV light. The consequences are major in the loss of cell cohesion and mechanical integrity. Certain enzymes involved in the terminal phases of keratinocyte differentiation play a key role in UV protection. Changes in the expression and/or activity of these enzymes have important consequences for the integrity of the barrier function and the homeostasis of the skin.

The extract of lavender aerial parts of the invention has demonstrated its effectiveness in reinforcing the barrier function and thus improving the protection of the skin against external aggressions (UV radiation, pollution, microorganisms, etc.).

The extract of lavender aerial parts of the invention has been shown to be effective in decreasing the level of melanin in skin biopsies, and thus inducing a lightening effect.

### EXAMPLES

By way of illustration, exemplary embodiments of the process according to the invention are described below.

### Example 1: Preparation of an Extract of Lavender (Lavandula angustifolia) of the Lamiaceae Family, Enriched with Small RNAs

### Extraction Process

Lavender flowers of the species Lavandula angustifolia are pre-dried in a ventilated place, protected from light. In a first step, 3% of dried lavender flowers and flower-bearing stems are ground into a powder with a particle size ranging from 500 μm to 1 mm, preferably 800 μm, i.e. the equivalent of 30 g of dried lavender flower powder in 968 g of distilled water. Then, 2 g/L or 3 mM of phytic acid is added. The pH is adjusted to 10.8 for optimal enrichment of the extract with low molecular weight RNAs.

The mixture is then heated for 1 hour at 80° C. with stirring.

After this hour of extraction, diatomaceous earths are added to the mixture in order to facilitate the subsequent separation between the solid residual plant material and the extract (soluble fraction).

The mixture is then filtered through filters with a pore size of 30 μm to remove the solid matter. The pH is then adjusted to pH 7.5 using an HCl solution. Sequential filtrations on filters of decreasing porosity are then carried out in order to clarify the plant extract until a sterilising filtration at 0.2 μm.

At the end of this step, the pH is checked and then adjusted to 6.3 with an HCl solution. pH values of 6 and 6.5 preserve the small RNAs in the extract.

An aqueous extract with a dry weight of 12.6 g/kg is obtained.

### Characterisation of the Lavender Extract

The extract obtained has a dry weight of 12.6 g/kg.

The physicochemical analysis shows that the extract obtained has a concentration of 3.7 g/kg of total sugars, 1160 g/kg of total organic acids, 1270 mg/kg of total phenolic compounds and 118 mg/kg of low molecular weight RNAs with a maximum length of 150 nucleotides.

The extract is then diluted with a cosmetic solvent that is physiologically acceptable and makes it possible to ensure better stability and better conservation of the extract over time.

The dilution is carried out with plant-derived butylene glycol to obtain a final concentration of 50% butylene glycol and 50% lavender extract. The diluted extract then has a dry weight of 6 g/kg and has a concentration of 1.7 g/kg total sugars, 570 mg/kg total organic acids, 620 mg/kg total phenolics and 45 mg/kg low molecular weight RNAs with a length of at most 150 nucleotides.

### Assay Methods Used to Determine the Amount of Different Compounds Contained in the Final Lavender Extract:

The total sugar content of the extract was determined by spectrophotometric assay based on an adaptation of the assay described by Dubois et al (1956) (Dubois et al, “Colorimetric method for the determination of sugars and related substances”, Anal. Chem, 1956, 28 (3), 350-356). This analysis consists of dissolving the raw material in concentrated sulphuric acid and then reacting it with phenol to form a coloured complex. The absorbance of the complex is read on the spectrophotometer at 490 nm. The sugar content was determined using a standard glucose curve. A TLC analysis showed that the majority of the sugars present in the extract of the invention are glucose and fructose molecules as well as high molecular weight sugars (oligo and polysaccharides).

The total polyphenol content of the lavender extract was determined by the Folin-Ciocalteu spectrophotometric assay method (Singleton et al., Analysis of total phenols and other oxidative and antioxidant substrates by means of the Folin-Ciocalteu reagent, 1999, 299: 152). Polyphenol compounds in the sample react with the Folin-Ciocalteu reagent; the oxidation of the reagent gives a blue colour. The absorbance of the sample is read on the spectrophotometer at 760 nm. The content is expressed in gallic acid equivalents using a gallic acid standard curve.

The characterisation of organic acids was performed on the Lavender extract from Example 1, a floral water and a reference essential oil. High-performance liquid chromatographic analysis, coupled with a mass detector was used. All samples were separated on an EC 150/4.6 Nucleoshell RP 18plus-5 μm column (150×4.6 mm) (Macherey Nagel: 763236.46) by an Agilent 1260 HPLC system (Agilent Technologies). The flow rate was 0.3 ml/min. The mobile phase consisted of a 0.01% solution of formic acid (HCOOH) (A) and acetonitrile (B). The gradient program facilitated elution as described in Table 2.

TABLE 2

| | A | B |
|---|---|---|
| | 0.01% | Acetonitrile |
| Time | HCOOH | (ACN) |
| (min) | (%) | (%) |
| 0 | 100 | 0 |
| 9 | 100 | 0 |
| 13 | 5 | 95 |
| 14 | 5 | 95 |
| 15 | 100 | 0 |
| 20 | 100 | 0 |

The column temperature was maintained at 25° C. and the injection volume was 5 μL. Detection was performed by an ACQUITY Qda mass spectrometer detector (WATERS) with an electrospray ion source in negative mode. The source was set at a capillary voltage of 0.8 kV and a probe temperature of 600° C.

M/z and cone voltage were targeted for each compound as described in Table 3.

TABLE 3

| | | Cone |
|---|---|---|
| | Mass | voltage |
| Compounds | (m/z) | (V) |
| Tartaric acid | 149.0 | 10 |
| Malic acid | 132.9 | 3 |
| Citric acid | 191.0 | 10 |
| Lactic acid | 88.9 | 5 |
| Succinic acid | 116.95 | 15 |
| Uronic acid | 193 | 10 |

The identification of organic acids was performed by comparing the retention times and mass spectral peaks of the sample with a standard. Quantitative estimation of organic acids was carried out on the basis of the maximum surface of the sample concentrations compared to the maximum surface of the standards.

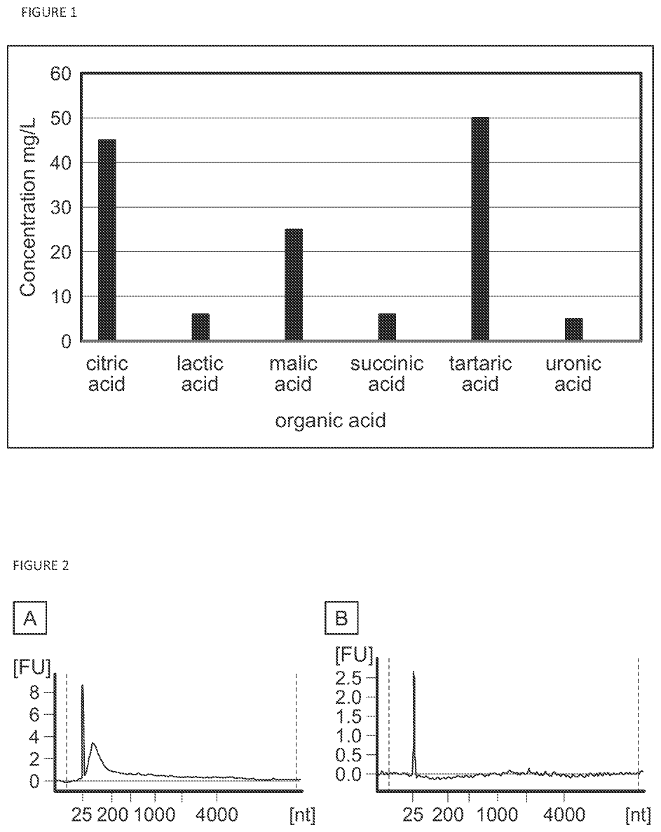

The HPLC-MS analysis, which allows the quantification and identification of the organic acids contained in the extract, shows that only the lavender extract contains different types of organic acids, mainly citric, malic and tartaric acid, as presented in FIG. 1. HPLC-MS analysis shows that these organic acids are not present in either the true lavender floral water or the true lavender essential oil.

The quantification of low molecular weight RNAs was carried out using a miniaturised electrophoresis technique on microfluidic chips specific to the analysis of nucleic acids such as low molecular weight RNAs (Bioanalyser 2100@, Agilent). This method makes it possible to determine the size and concentration of nucleic acids contained in an extract from a few microlitres. The result is presented as a graph with an arbitrary fluorescence unit (FU) on the ordinate and the number of nucleotides (nt) on the abscissa. An internal marker is added to each analysis (peak at 25 nt in FIG. 2), and serves as an internal control to validate the correctness of the analysis.

FIG. 2 shows the analysis of low molecular weight RNAs by the 2100 Bioanalyzer. A: Lavender extract according to example 1. B: conventional lavender extract according to example 2.

Bioanalytical analysis shows that the process described in the present invention is capable of extracting low molecular weight RNAs from lavender, as illustrated in FIG. 2A. FIG. 2A shows that the RNAs present in the lavender extract of the invention have molecular weights ranging from greater than 25 to about 150 nucleotides.

A conventional extraction process such as maceration described below in example 2 does not extract low molecular weight RNAs (FIG. 2B). The analysis also showed that these molecules are not present either in the floral water or in the essential oil. Moreover, this analysis demonstrates the absence of DNA in the extract.

The analysis of volatile odorous compounds (VOC) was performed on the lavender extract according to the invention, the floral water and the essential oil by GC-FID. All samples were separated on a 30 mx 250 μm×0.25 μm GC OPTIMA 5HT column (Macherey-Nagel 726106.30) by Agilent GC/FID 7890A gas chromatography (Agilent Technologies). 3 μL of sample products were injected onto the column at a flow rate of 1.3 ml/min, using helium as the carrier gas on a programmed oven that rises from 75° C. to 320° C. in 40 min.

The molecules were detected on a flame ionisation detector at 230° C., using hydrogen (30 ml/min) and air (400 ml/min) for the flame. The identification of the VOCs is done by comparing the retention times of the compounds.

For aqueous samples, a liquid/liquid extraction in hexane (1:1) is performed prior to injection. Magnesium sulphate is added to the organic phase in order to remove all traces of water.

TABLE 4
Determination of volatile odorous compounds in lavender extract

| Volatile odorous | LD | Lavender |
|---|---|---|
| molecules (VOCs) | (ppm) | extract |
| Eucalyptol | 5 | <LD |
| Thujones | 3 | <LD |
| Linalool | 4 | <LD |
| Camphor | 2 | <LD |
| Borneol | 3 | <LD |
| Lynalyl acetate | 3 | <LD |

The results of the GC-FID analysis in Table 4 show that the lavender extract in example 1 does not contain any odorous molecules of a terpene nature, unlike lavender floral water and essential oil, which are known to contain these molecules in majority.

### Example 2 Preparation of a Lavender Macerate

In order to compare a so-called classical extraction to the extract of the invention, a lavender macerate was made, using the same quantity of dried lavender flowers of the species Lavandula angustifolia as in example 1, i.e. 3% of crushed dried lavender flowers in distilled water. The mixture is then heated for 1 hour at 80° C., then the mixture is filtered by a first filtration with a large porosity of 30 μm in order to remove the solid residual plant matter from the liquid part, then by sequential filtration with decreasing porosity down to 0.2 μm. The purpose of this process is to prepare a control extract to obtain comparative analytical data with respect to the lavender extract obtained by the process of the invention. The results obtained are illustrated in FIG. 2B and in the text of the application.

### Example 3: Evaluation of the Lavandula angustifolia Extract of Example 1 on Reactive Oxygen Species after Application of Visible Light Stress on Normal Human Keratinocytes

Principle:

The aim of this study is to show the effect of the lavender extract prepared according to example 1 on the decrease of reactive oxygen species generated by visible light stress. This type of light between 400 and 700 nm is intended to mimic daylight. Reactive oxygen species are involved in various mechanisms of protein and DNA change, related to skin ageing.

Protocol:

Normal human keratinocytes are treated with the extract from example 1 overnight. The cells are then exposed to visible light generated by a 24 Watt, 5000° Kelvin spotlight, to mimic daylight. This exposure is repeated 4 times for 10 minutes during the day. The treatment is applied again during the night, and then the cells are again subjected to the same visible light stress. At the end of this stress, reactive oxygen species are detected by the mitochondrial probe MitoSOX™ Red (ThermoFisher scientific).

Results:

The application of daylight stress resulted in an increase in reactive oxygen species (ROS) of +43% compared to unexposed cells. When the cells were treated with the extract of example 1 at 0.1% (volume/volume dilution), ROS decreased highly significantly by −12% compared to untreated cells. A lavender macerate obtained according to example 2 and tested under the same conditions resulted in a smaller decrease of −5%.

Conclusion:

Lavender extract showed antioxidant activity, directed against reactive oxygen species at the mitochondrial level. This activity was found to be higher than that obtained with lavender extract from a conventional maceration.

### Example 4: Evaluation of the Extract of Example 1 on DNA Damage in Normal Human Melanocytes Exposed to UVB Stress

Principle:

Genomic instability can be defined as all the chemical modifications of DNA, which occur during different processes and can accumulate over time. DNA changes are a major component of photoaging. In the present study, we focused on UVB damage in melanocytes.

Pyrimidine dimers result from a direct effect of UVB on the pyrimidine bases of DNA. Cyclobutane pyrimidine dimers (CPD) are formed and are the most common form of UVB-induced DNA damage. In melanocytes, CPDs continue to be generated for more than 3 hours after UVB exposure. They are called “dark CPDs” and are due to the chemiexcitation of melanin, leading to an energy transfer to DNA.

In this study, the ability of the extract in example 1 to reduce the formation of dark CPDs in melanocytes was evaluated.

Protocol:

Melanocytes extracted from human epidermis are treated with the extract of example 1 at 0.1% (volume/volume dilution) overnight, then irradiated with UVB at 60 mJ/cm2 and treated again overnight with the lavender extract. The next morning, pyrimidine dimers were detected by immunostaining with an antibody against CPDs (Cyclobutane Pyrimidine Dimers Mouse Monoclonal, Euromedex). After one and a half hours of incubation followed by rinses, the cells are incubated in the presence of the anti-mouse secondary antibody coupled to a fluorophore (Alexa Fluor® 488, Invitrogen). The cells are then examined under an epi-fluorescence microscope (Zeiss Axiovert 200M microscope). The presence of CPDs is then observed and quantified by image analysis (Volocity® image analysis software, Improvision).

Results:

Under non-irradiated conditions, melanocytes do not exhibit CPDs. Their formation is induced as a result of UVB stress. Application of the extract from example 1 to melanocytes reduced the induction of dark CPDs by UVB by −37% (highly significant by Student's t-test compared to untreated irradiated cells). Under the same conditions and at the same concentration of 0.1%, the lavender macerate obtained according to example 2 had no significant effect.

Conclusion:

The extract in example 1 decreased the “dark CPDs” produced in melanocytes by UVB stress. Through this activity, the lavender extract showed a benefit in reducing DNA damage, contributing to the skin's repair mechanisms during the night.

### Example 5: Evaluation of the Extract of Example 1 on the Melatonin Synthesis Pathway in Ex Vivo Skin Biopsies and Human Hair Follicles

Principle:

The aim of this experiment is to demonstrate an effect of the extract from example 1 on melatonin synthesis in cultured human skin biopsies. This evaluation includes two markers: 1—the enzyme AANAT (aralkylamine N-acetyltransferase or serotonin N-acetyltransferase or timezyme), which catalyses the N-acetylation of serotonin to N-acetylserotonin, the final step in the synthesis of melatonin, 2—the evaluation of melatonin itself. The AANAT enzyme controls the day/night rhythm of melatonin production in the pineal gland. As melatonin is also synthesised locally in the skin, it was sought to monitor its synthesis in response to the application of lavender extract.

Protocol:

The enzyme AANAT and melatonin are assessed by indirect immunofluorescence on skin biopsies, pre-treated by topical application of lavender extract for 48 hours (twice daily). In addition, the extract of Example 1 diluted to 0.5% (volume/volume dilution) in the culture medium was contacted with human hair follicles isolated from scalp biopsies. Control biopsies incubated in parallel under the same conditions, as well as control hair follicles without the addition of lavender extract, were given the placebo (Phosphate Buffer Saline, PBS). After incubation, the biopsies and hair follicles are fixed and embedded in paraffin in order to produce histological sections. The detection of the enzyme AANAT and melatonin is performed by incubation with the respective antibodies: anti-AANAT (Invitrogen) and anti-melatonin (Abnova, Cliniscience). After one and a half hours of incubation followed by rinses, the sections are incubated in the presence of the anti-rabbit secondary antibody coupled to a fluorophore (Alexa Fluor® 488, Invitrogen). The sections are then examined under an epi-fluorescence microscope (Zeiss Axiovert 200M microscope). Collagen I expression is then observed and quantified by image analysis (Volocity® image analysis software, Improvision).

Result:

Assessment of the enzyme AANAT and melatonin showed an increase of +20% and +51%, respectively, in skin biopsies treated with the 0.5% extract of example 1 (highly significant by Student's t-test, compared to biopsies receiving placebo). The lavender macerate obtained according to example 2 gave an equivalent result for AANAT, but led to a smaller increase for melatonin (+34%, highly significant by Student's t-test, compared to biopsies receiving placebo). In cultured hair follicles, the 0.5% extract from example 1 resulted in a +23% increase in melatonin production, observed in the outer epithelial sheath of the follicles.

Conclusion:

The extract from example 1 showed activity on melatonin production in ex vivo skin biopsies and in hair follicles. This increase is related to an increase in the enzyme AANAT, the expression of which is increased in the pineal gland during the transition from day to night. The properties of melatonin are related to cellular damage repair activities, especially in DNA and due to its antioxidant activity. The increase of melatonin in the skin by lavender extract therefore appears to be beneficial for the skin's damage repair processes during the night. The increase in melatonin in the hair follicle indicates a beneficial effect on hair follicle physiology, as melatonin is associated with the hair growth phase.

### Example 6: Evaluation of the Lightening Potential of the Extract of Example 1 on Ex Vivo Skin Biopsies

Principle:

The aim of this study is to evaluate the lightening potential of the extract of example 1 on ex vivo skin biopsies, using the histological staining of melanin Fontana-Masson, based on the reduction of an ammoniacal silver nitrate solution to metallic silver. The staining obtained reveals the melanin content and is quantified by image analysis.

Protocol:

Ex vivo human skin biopsies are cultured and treated with the extract of example 1 at 0.5% (volume/volume dilution) and 1% (volume/volume dilution) for 48 hours. After treatment, the biopsies are fixed for histological analysis and embedded in paraffin. After deparaffinisation, the sections are incubated with ammoniacal silver nitrate solution at 60° C. for 10 minutes. After rinsing, they are treated with 5% sodium thiosulphate for 2 minutes, rinsed again and mounted for examination under the Eclipse E600 microscope (Nikon). The pictures are taken with the QImaging Retiga 2000R Fast1394 camera and analysed with Q-Capture Pro 7 software (QImaging).

### Results and Conclusion

In ex vivo skin biopsies, a decrease in melanin content of minus 55% and minus 72% was observed after application of the extract of Example 1 at 0.5% (volume/volume dilution) and 1% (volume/volume dilution) respectively (highly significant by Student's t-test compared to placebo biopsies), whereas the lavender macerate obtained according to example 2 showed no decrease at 0.5%, and a smaller decrease (of −47%) at 1%.

This test therefore concluded that there was a potential lightening effect of the Lavandula angustifolia extract of example 1 on ex vivo skin biopsies.

### Example 7: Formula for a Rich Cream

TABLE 5

| | | |
|---|---|---|
| Ingredients (Brand name) | INCI | % w/w |

Phase A

| | | |
|---|---|---|
| Purified water | Aqua | q.s. 100 |
| Optiphen ™ Plus preservative | Phenoxyethanol (and) Caprylyl Glycol (and) | 1.50 |
| | Sorbic Acid | |

Phase B

| | | |
|---|---|---|
| Stabileze ™ QM polymer | PVM/MA Decadiene Crosspolymer | 0.15 |

Phase C

| | | |
|---|---|---|
| ProLipid ™ 141 lamellar gel | Glyceryl Stearate (and) Behenyl Alcohol (and) | 5.00 |
| | Palmitic Acid (and) Stearic Acid (and) Lecithin | |
| | (and) Lauryl Alcohol (and) Myristyl Alcohol (and) | |
| | Cetyl Alcohol | |
| Ceraphyl ™ 494 ester | Isocetyl Stearate | 4.00 |
| Ceraphyl ™ SLK ester | Isodecyl Neopentanoate | 4.00 |
| DC 580 Wax | Stearoxytrimethylsilane (and) Stearyl Alcohol | 2.00 |
| Emulsynt ™ GDL ester | Glyceryl Dilaurate | 3.00 |

Phase D

| | | |
|---|---|---|
| Gransil DM-5 | Dimethicone (and) Polysilicone-11 | 3.00 |

Phase E

| | | |
|---|---|---|
| Sodium hydroxide | Sodium Hydroxide | 0.04 |
| Purified water | Aqua | 0.50 |

Phase F

| | | |
|---|---|---|
| PF Precious Wood | Perfume/Fragrance | 0.30 |
| Unipure* Red LC 381 ADT-C | CI 77491 (Iron oxides) (and) Isopropyl Titanium | 0.03 |
| | Triisostearate (and) Bis-Hydroxyethoxypropyl | |
| | Dimethicone (and) PEG-2-Soyamine (and) | |
| | Isophorone Diisocyanate | |

Phase G

| | | |
|---|---|---|
| Extract according to example 1 | Butylene glycol (and) Lavandula Angustifolia | 3.00 |
| | Flower Extract | |
| Ronaflair Balance Gold | CI 77891 (Titanium Dioxide) (and) Mica (and) Tin | 0.30 |
| | Oxide | |
| Covabead Velvet 10 | Polymethyl Methacrylate | 1.00 |
| Ronaflair Balance Red | CI 77891 (Titanium Dioxide) (and) Mica (and) Tin | 1.20 |
| | Oxide | |

Phase H

| | | |
|---|---|---|
| Purified water | Aqua | 15.00 |
| Natrosol ™ Plus 330 CS | Cetyl Hydroxyethylcellulose | 0.50 |

Preparation Process:
1. homogenise phase A in the main vessel and start heating to 75-80° C.;
2. at 30° C., sprinkle in phase B and homogenise while heating;
3. in a separate beaker, prepare phase C, heat at 75-80° C. until homogeneous;
4. at 75° C., add phase C to the main vessel and homogenise for 10 minutes;
5. allow the temperature to cool and add phase D at 65° C. Mix well to homogenise for 10 minutes;
6. pre-mix phase E before adding it to the main vessel;
7. add phase E at 60° C. Mix well to homogenise for 10 minutes;
8. at 35° C., pre-mix phase F before adding and mixing well;
9. pre-mix phase G before adding it to the main vessel;
10. add phase G at 35° C. Mix well to homogenise;
11. in a separate beaker, prepare phase H: sprinkle Natrosol™ into room temperature water and homogenise while heating to 60° C.;
12. add phase H at 30° C. Mix well to homogenise;
13. stop at 25° C.

The composition is thus in the form of a pink buttercream, with a pH between 4.90 and 5.40 and a viscosity (DO) of 160,000-210,000 cps (Brookfield RVT/spindle D/5 RPM/1 minute/25° C.).

### Example 8: Anti-Ageing Mask Formula

TABLE 6

| | | |
|---|---|---|
| Ingredients (Brand name) | INCI | % w/w |

Phase A

| | | |
|---|---|---|
| Purified water | Aqua | q.s. 100 |
| Tetrasodium EDTA | Tetrasodium EDTA | 0.05 |

Phase B

| | | |
|---|---|---|
| N-Hance ™ HP40S guar | Hydroxypropyl Guar | 0.10 |

Phase C

| | | |
|---|---|---|
| Lubrajel ™ DV Free hydrogel | Glycerin (and) Glyceryl Acrylate/Acrylic Acid | 6.00 |
| | Copolymer | |

Phase D

| | | |
|---|---|---|
| Si-Tec ™ GF 3096 silicone | Dimethicone (and) Dimethiconol | 12.00 |
| RapiThix ™ A-60 polymer | Sodium Polyacrylate (and) Hydrogenated | 2.40 |
| | Polydecene (and) Trideceth-6 | |

Phase E

| | | |
|---|---|---|
| Optiphen ™ Plus preservative | Phenoxyethanol (and) Caprylyl Glycol (and) Sorbic | 1.50 |
| | Acid | |

Phase F

| | | |
|---|---|---|
| Surfin* 96 | Alcohol Denat. | 3.50 |
| PF Cucumber & Aloe | Perfume/Fragrance | 0.50 |

Phase G

| | | |
|---|---|---|
| Extract according to example 1 | Butylene glycol (and) Lavandula Angustifolia Flower | 1.00 |
| | Extract | |
| Achromaxyl ™ ISR | Water/Aqua (and) Glycerin (and) Hydrolysed | 3.00 |
| biofunctional | Brassica Napus Seedcake Extract | |
| Xirona Carribean Blue | Mica (and) CI 77891 (Titanium Dioxide) (and) Silica | 1.00 |
| | (and) Tin Oxide | |

Preparation Process:
1. at 25° C., homogenise phase A in the main container;
2. at 25° C., sprinkle in phase B and mix well until homogeneous;
3. at 25° C., add phase C and mix well until homogeneous;
4. premix phase D in a separate beaker and add to the main vessel at 25° C.;
5. at 25° C., add phase E to the main vessel and mix well;
6. pre-mix phase F and add it slowly. Mix well until homogeneous;
7. pre-mix phase G in a separate beaker and add to the main vessel until homogeneous;
8. stop at 25° C.

The composition is thus presented as a creamy gel with glittering green effects, with a pH between 5.30 and 5.80 and a viscosity (DO) of 70,000-100,000 cps (Brookfield RVT/spindle C/5 RPM/1 minute/25° C.).

### Example 9: Serum Formula

TABLE 7

| | | |
|---|---|---|
| Ingredients (Brand name) | INCI | % w/w |

Phase A

| | | |
|---|---|---|
| Demineralised water | Aqua | 87.40 |
| Sodium Hyaluronate | Sodium Hyaluronate | 0.20 |
| RapiThix ™ A-60 polymer | Sodium Polyacrylate (and) | 0.40 |
| | Hydrogenated Polydecene (and) | |
| | Trideceth-6 | |
| Lubrajel ™ DV hydrogel | Glycerin (and) Glyceryl | 6.00 |
| | Acrylate/Acrylic Acid Copolymer (and) | |
| | Propylene Glycol | |
| Lubrajel ™ Oil hydrogel | Glycerin (and) Glyceryl | 1.00 |
| | Acrylate/Acrylic Acid Copolymer (and) | |
| | Propylene Glycol (and) PVM/MA | |
| | Copolymer | |
| Wacker-Belsil* DM 100 | Dimethicone | 2.00 |
| Cyclopentasiloxane NF | Cyclopentasiloxane | 0.50 |
| Extract according to example 1 | Butylene glycol (and) Lavandula | 1.00 |
| | Angustifolia Flower Extract | |
| Optiphen ™ preservative | Phenoxyethanol (and) Caprylyl Glycol | 1.50 |

Preparation process:
1. Add Water to the Main Container and Start Mixing with a Hi-Lo Propeller Blade;
2. add the rest of the ingredients, one after the other, while mixing between each addition.

The composition is thus a smooth, semi-opaque serum with a pH between 5.75 and 6.25 and a viscosity (DO) of 1,100-1,400 cps (Brookfield RVT/spindle 3/20 rpm/25° C./1 minute).

What is claimed is:

1. A process for obtaining an aqueous extract of lavender aerial parts, comprising the following steps
a) bringing the lavender aerial parts into contact with water;
b) adding phytic acid at a concentration of between 1 and 10 mM to the mixture obtained in a) at a pH of between 10 and 11;
c) then adjusting the pH of the mixture obtained in b) to a value of between 6 and 8;
d) purifying the mixture obtained in c) so as to eliminate the residual solid plant matter and obtain a purified aqueous crude extract; and
e) checking the pH and readjusting it if necessary to a value between 6 and 8, preferably between 6 and 6.5.

2. The process according to claim 1, characterised in that in step a) the lavender aerial parts, previously dried and then ground, are brought into contact with water in a plant material/water ratio of between 3 and 20% (weight/weight).

3. The process according to claim 1, characterised in that step b) treatment with phytic acid at a concentration of 3 mM is carried out with stirring for a time of at least 1 hour and at a temperature of between 20 and 80° C.

4. The process according to claim 1, characterised in that step e) is preceded by at least one filtration of the aqueous crude extract obtained in d) and preferably by successive filtrations of the aqueous crude extract by lowering the filtration threshold from 20-50 μm to 0.10-0.30 μm.

5. The process according to claim 1, characterised in that the lavender aerial parts are of the species Lavandula angustifolia.

6. An aqueous extract of lavender aerial parts enriched with small RNAs of a length of at most 150 nucleotides, with sugars, with phenolic compounds and with organic acids, free of DNA, obtainable by the process of claim 1, characterised in that it comprises, by weight of the total weight of the extract, 10 to 30 g/kg of dry weight, containing 2 to 10 g/kg of sugars, 100 to 1500 mg/kg of organic acids, 500 to 2000 mg/kg of phenolic compounds and 40 to 200 mg/kg of low molecular weight RNAs, with a length of at most 150 nucleotides.

7. The extract according to claim 6, characterised in that it is subsequently diluted in a solvent and comprises, by weight of the total weight of the extract, 4 to 20 g/kg of dry extract, 0.5 to 10 g/kg of sugars, 50 to 700 mg/kg of organic acids, 50 to 1500 mg/kg of phenolic compounds and 10 to 100 mg/kg of low molecular weight RNAs with a length of at most 150 nucleotides.

8. A composition comprising, as active ingredient, an effective amount of the extract of claim 6, and a physiologically acceptable medium.

9. The composition according to claim 8, characterised in that the extract is present at a concentration of between 0.05 and 5% by weight of the total weight of the composition and preferably between 0.1 and 1.0% by weight of the total weight of the composition.

10. The composition according to claim 8, characterised in that it is formulated to be applied topically to the skin, skin appendages and scalp.

11. Cosmetic use of the composition of claim 10 for skincare, care of the scalp and the skin appendages, and more particularly for protecting the skin from external aggressions and oxidation, combating the signs of skin ageing, increasing photoprotection, lightening the skin, improving skin hydration, reinforcing the barrier function, or soothing the skin.

12. Cosmetic use of the composition of claim 10 for improving biological mechanisms associated with skin repair during the night.
